(19) United States
(12) Patent Application Publication
(10) Pub. No.: US 2023/0164287 A1
(43) Pub. Date: May 25, 2023

(54) IMAGING METHOD FOR IMAGING A SCENE AND A SYSTEM THEREFOR

(71) Applicant: KARL STORZ SE & Co. KG, Tuttlingen (DE)

(72) Inventor: Bernd Steinke, Schaffhausen (CH)

(73) Assignee: KARL STORZ SE & Co. KG, Tuttlingen (DE)

(21) Appl. No.: 18/056,797

(22) Filed: Nov. 18, 2022

(30) Foreign Application Priority Data
Nov. 22, 2021 (DE) 10 2021 130 529.2

(51) Int. Cl. H04N 5/33; G01S 17/89

(52) U.S. Cl. CPC H04N 5/33; G01S 17/89; H04N 5/2256

(57) ABSTRACT

Methods (10, 10′, 10″) for imaging a scene (110) during a change of a field of view (112) of an image sensor (114) relative to the scene (110) and a corresponding system (100) are disclosed.

### CROSS REFERENCE TO RELATED APPLICATIONS

This application claims priority to German Patent Application No. 102021130529.2, filed Nov. 22, 2021, and entitled, “Bildgebendes Verfahren zum Abbilden einer Szene and System,” and is in its entirety incorporated herein by reference.

### TECHNICAL FIELD OF THE INVENTION

The invention relates to an imaging method for imaging a scene during a change of a field of view of an image sensor relative to the scene. The invention further relates to a system for imaging a scene during a change of a field of view of an image sensor relative to the scene.

### BACKGROUND OF THE INVENTION

As is evident from the explanations below, the invention offers advantages in applications where image information from two different spectral ranges should be processed. For the explanations below, these spectral ranges will be described as the white light range and the near infrared (NIR) range. However, the invention is not restricted to these spectral ranges. Moreover, images obtained in endoscopy are discussed in exemplary fashion. However, the invention is not restricted to this type of images.

Imaging applications making use of image information from two different, distinct spectral ranges arises, for example, within the scope of imaging with the aid of near infrared fluorescence. For examples of methods and products making use of such imaging techniques see https://www.karlstorz.com/ro/en/nir-icg-near-infrared-fluorescence.htm. These fluorescence imaging products and methods require very powerful light sources in the white light range and, even more essentially, the excitation radiation in the near infrared fluorescence range, wherein a scene is exposed to a NIR radiation in an excitation wavelength band that is absorbed by a given fluorophore present in the scene. The excited fluorophore then emits a fluorescent emission radiation of a longer wavelength. In general, the emission radiation has a much lower intensity than that of the excitation radiation, and therefore it is important to provide as much excitation illumination as is practical. Usually, FI imagery is accompanied by white light imagery, and the two image streams are overlayed. In the case of NIR FI imagery, the FI image is usually represented as a false color in the visible range overlaid on the white light image.

This need for increased NIR intensity is due to several additional factors including throughput restrictions of the fluorescence light system. When operating in an alternating white light/fluorescence light mode, which is commonly the case, fewer frames in a given time period are collected for each in a respective stream of white light and fluorescence light images. As a result of operating in this alternating illumination mode, shorter exposure times may be required in order to obtain a desired frame rate. Finally, wait times are required when a rolling shutter is used (as is the usually the case with common CMOS sensors), in order to avoid erroneous frames that might, for example, exhibit partial superpositions of white light and fluorescence light further decreasing the possible frame rate.

As a consequence, fluorescence images usually have a lower contrast and/or look washed out. The relatively weak fluorescence effects are less accurately recognizable in this case. A resultant NIR emission image is usually displayed as a false-colored green glow and has a significantly lower contrast and fewer details than a corresponding white light image. However, the NIR image contains information that plays an important role, especially in the medical field, enabling the visualization of elements that are otherwise not recognizable and/or visible in a white light image.

The most common conventional means for producing NIR excitation light is with the use of high power xenon lamps. However, these lamps have a number of disadvantages, including a relatively short service life with early degeneration and significant noise due to the powerful ventilation required in order to keep the illumination systems from overheating. An additional reason for the inefficiency of xenon lamps for FI excitation illumination is that, while they produce a wide wavelength band, only the NIR light component is used as a fluorescence source. The additionally produced powerful white light component is removed by optical filtering which must be dissipated as heat. Additionally, xenon lamps themselves produce relevant waste heat in normal operation.

Therefore, it is becoming increasingly more common to make use of LEDs, which offer less waste heat, resulting in reduced ventilator noise, and offer an increased lamp service life. However, the limited IR luminous intensity of the LED requires a comparatively greater amplification of the detected image signal, for example by means of automatic gain control (AGC). However, this amplification has the disadvantage of also amplifying noise, and hence a introducing a certain blurriness/unsharpness. If this noise is attempted to be removed by averaging, a reduced contrast in the fluorescence channel can result. This effect can be partially counteracted by way of a longer exposure. However, longer exposure times can lead to images of compromised quality and blurriness as a result of even the slightest movement of the image capturing system, for example a slight camera shake.

Finally, laser-based solutions are also used. However, since the use of lasers, particularly high powered lasers, may require laser protection goggles, lasers are generally only used when all other options are unable to deliver the desired results.

In order to obtain the desired imaging result, an increased level of detail, less image noise, and a higher contrast are desired. Such desired imaging quality can facilitate the physician's interpretation of an endoscopic image, for example, in relation to regions of the image with insufficient blood perfusion, a common application of the fluorophore indocyanine green (ICG). Thus, a reliable distinction can be made between well perfused regions and tissue portions insufficiently connected to the blood supply, especially in the case of sharp boundaries that are reliably identifiable with a good dynamic range. This likewise applies for the identification of cancerous tissue regions in contrast to healthy tissue.

The present invention discloses an improved imaging method and a corresponding system for imaging a scene during a change in a field of view of an image sensor relative to the scene.

### SUMMARY OF THE INVENTION

According to one aspect, an improved imaging method for imaging a scene during a change of a field of view of an image sensor relative to the scene is presented, the method including the following steps:
a) capturing a first image in the scene with a first illumination of the scene at a first time, with the first image predominantly containing first image information from the scene in a visible light range,
b) subsequent to step a, capturing a plurality of second images in the scene with a second illumination of the scene at a respective second time, wherein the second images predominantly containing second image information from the scene in a non-visible light range,
c) subsequent to step b, capturing a third image in the scene with the first illumination of the scene at a third time, with the third image predominantly containing third image information from the scene in the visible light range,
d) determining at least one reference feature in the scene, where the reference feature is imaged in the first and in the third image,
e) determining a first change of the field of view on the basis of the at least one reference feature,
f) registering the second images while considering at least one second change, which arises as a first proportion of the first change, with the first proportion being the ratio of a first time difference between the second times of two second images to be registered and a second time difference between the third time and the first time,
g) processing the registered second images in order to obtain a resultant image, and
h) outputting the resultant image.

In a preferred method, the method disclosed above includes the further steps of:
i) subsequent to step b and before step c, capturing a fourth image in the scene with the first illumination of the scene at a fourth time, with the fourth image predominantly containing fourth image information from the scene in the visible light range, subsequent to step i and before step c, capturing a plurality of fifth images in the scene with the second illumination of the scene at a respective fifth time, with the fifth images predominantly containing fifth image information from the scene in the non-visible light range,
k) subsequent to step e and before step g, registering the fifth images while considering at least one third change, which arises as a second proportion of the first change, with the second proportion being the ratio of a third time difference between the fifth times of two fifth images to be registered and the second time difference between the third time and the first time, and
with the registered second images and the registered fifth images being processed in step g in order to obtain a resultant image.

This method allows even more in-depth registration of images with one another, which predominantly contain image information in a non-visible light range. In the process, rolling registration of images is also possible, for example initially registering a first-second image with a second-second image, then the second-second image with a first-fifth image, then the first-fifth image with a second-fifth image, etc.

In a another, alternative preferred configuration,
step d is instead carried out as follows: determining at least one first reference feature in the scene, which first reference feature is imaged in the first and in the fourth image, and at least one second reference feature in the scene, which second reference feature is imaged in the fourth and in the third image,
step e is instead carried out as follows: determining a first change of the field of view on the basis of the at least one first reference feature and a further change of the field of view on the basis of the at least one second reference feature, and
step f is instead carried out as follows: registering the second images while considering at least one second change, which arises as a first proportion of the first change, with the first proportion being the ratio of a first time difference between the second times of two second images to be registered and a second time difference between the fourth time and the first time,
step k is instead carried out as follows: registering the fifth images while considering at least one third change, which arises as a second proportion of the further change, with the second proportion being the ratio of a third time difference between the fifth times of two fifth images to be registered and a fourth time difference between the third time and the fourth time.

This method allows the fourth image to be used as supporting image for the registration of the second and fifth images. This can increase the accuracy within the scope of the registration.

In a preferred configuration, a first intensity of the first image and of the third image is greater in each case than a second intensity of each of the second images.

In this context, the second intensity may be no more than 50%, 33%, 25%, 20%, 15% or 10% of the first intensity, in particular. In particular, the intensity of an image can be determined as mean or median of all pixels.

In a preferred configuration, the second images predominantly contain second image information from the scene in a near infrared range.

This configuration is particularly suitable for the application in the field of fluorescence imaging.

In a preferred configuration, the second images are brought into correspondence with a reference image.

Hence, such a reference image can be an image from both the visible light range and the non-visible light range. While it is desirable in the former case to represent the images from the non-visible light range together with the images from the visible light range, for example as an overlay, it is desirable in the latter case to represent a separate image with image information from the non-visible light range.

In a preferred configuration, the processing of the second registered images includes the application of computational photography. Computational photography offers various options for representing the image information from the non-visible light range with a higher quality, for example with clearer contours. The application of computational photography is possible since the images can be registered with image information from the non-visible light range, even if the image information thereof itself does not allow a conventional registration or does not allow a conventional registration with sufficient accuracy. For more information on computational photography, see, for example, https://en.wikipedia.org/wiki/Computational_photography.

It is understood that the features mentioned above and the features yet to be explained below are applicable not only in the respectively specified combination but also in other combinations or on their own, without departing from the scope of the present invention.

### BRIEF DESCRIPTION OF THE DRAWINGS

Exemplary embodiments of the invention are depicted in the drawings and are described in more detail in the following description.

FIG. 1 shows a first embodiment of an imaging method.

FIG. 2 shows a first chronological sequence for recording images.

FIG. 3 shows a second chronological sequence for recording images.

FIG. 4 shows a third chronological sequence for recording images.

FIG. 5 shows a second embodiment of an imaging method.

FIG. 6 shows a third embodiment of an imaging method.

FIG. 7 shows a fourth chronological sequence for recording images.

FIG. 8 shows a fifth chronological sequence for recording images.

FIG. 9 shows a first embodiment of a system for imaging a scene.

FIG. 10 shows a second embodiment of a system for imaging a scene.

### DETAILED DESCRIPTION OF THE INVENTION

In the context of the present invention, the conventional means of improving the image quality of the second images, that predominantly contain second image information from the scene in a non-visible light range, are stretched to their limits. By way of example, more sensitive image chips for recording the second images can be significantly more expensive, and thus not desirable. Another potential solution, that of lengthening the exposure time for the second image leads to a blurring of contours on account of the change of the field of view of the image sensor, which can be caused by any movement of the image sensor relative to the scene. Another common solution, increasing the luminous intensity has only a limited effect, especially in the field of fluorescence imaging, since it is not reflected light that is sensed but rather the fluorescence emission from the fluorophore triggered by the excitation light. The terms “visible” and “non-visible” here relate to human vision, with “visible” describing a spectrum to which the human eye is sensitive and “non-visible” describing a spectrum to which the human eye is insensitive.

In the context of the present invention, directly overlaying a plurality of second images also does not lead to a satisfactory solution since this would also cause a “blurring” on account of the change in the field of view of the image sensor of each of the second images. Another considered solution is a direct registration (see, for example, https://de.wikipedia.org/wiki/Bildregistrierung or https://en.wikipedia.org/wiki/Image_registration) of the second images, but this was not found to be practical in all situations, especially if the second images cannot accurately be registered due a low contrast of the second images, for example.

Some of the benefits of the present invention arise from the field of view of the image sensor being determined on the basis of image information from images predominantly collected in the visible light range. In practice, this image information has sufficiently clear features able to be identified, and with the aid of these clearly identifiable features, the change in the field of view can be determined. In this context, the change can be determined from two immediately successive images with image information in the visible light range, or else from images which follow one another in time without being immediately successive.

It is possible to determine the times at which the images predominantly containing image information in a non-visible light range are captured, for example by reference a timing generator common in image capture systems. If the assumption is now made that the change in the field of view of the image sensor between the capture of images predominantly containing image information in the visible light range is at least substantially uniform, it is possible to calculate the extent to which the change has advanced at the time of recording of the images predominantly containing image information in the non-visible light range. Specifically, the change may include one or more elements of the group translation, rotation, tilt, change of an optical focal length or change of a digital magnification.

For example, if the assumption is made that a change A between a first time t1, at which time the first image is recorded, and a third time t3, at which time the third image is recorded, is T=t3−t1, and that a second image is recorded at a time t2=0.5 T, then the change between the first image and the second image is 0.5 A and it is also 0.5 A between the second image and the third image.

In another example, two second images are recorded at a time t2=0.4 T and t2′=0.6 T. Therefore, the change between the first-second image and the second-second image is T=t2′−t2=0.2 T, and so the change between the second images can be calculated as 0.2 A. If this example is expanded, it is possible to recognize that the second images can now be registered because the proportional change between the second images is known. Thus, it is not necessary to determine the change from the second images themselves. Although, this can be additionally implemented in order to increase the accuracy or to test reliability, it is not required to examine the second images themselves in respect to a change. Knowledge of the change in the corresponding images which predominantly contain image information in the visible light range allows the proportional change of the second images to be determined.

Further, the second images can also be registered to the first and/or the third image, since it is known in the above example that the change between the first image and the first second image is 0.4 A and the change between the second-second image and the third image is also 0.4 A. Thus, for example, the first-second image can be registered with the second-second image, or vice versa, and the resultant image can be registered with the first and/or the third image.

For the registration of the second images, each second image is assigned a corresponding second time; for example, this may be the second time at which the corresponding second image was recorded. In the case of slow changes and, in particular, if the intention is to register a plurality of images which predominantly contain image information in the non-visible light range, but which are between different images containing predominantly image information in the visible light range, it may be sufficient to assign the second images that are between the same images containing predominantly image information in the visible light range a certain second time, without assigning each second image an individual second time.

It should be noted that it is not necessary to process all images that predominantly contain image information in the non-visible light range. Rather, a selection can be made, and so only a subset of the recorded images represents the second images which are brought into correspondence. Accordingly, the second frames for processing can also be a subset of all second frames. Finally, not all second times have to be different.

The proposed solution is the optimization of both the white light image and, in preferred circumstances, the fluorescence light image. The fact that both images correlate alternately in a defined chronological sequence may be exploited by using the effects of this bi-spectral time offset for image improvement. In this way, the conventional “brute force” methods to improve FI overlay imaging by the amplification of the light source, particularly when LEDs are used, or amplification of the camera sensor signal, and all the accompanying deleterious effects and costs that accompany such brute force methods, can be avoided. Instead, the present invention makes innovative use of video processing algorithms for fluorescence light recordings, including a correction using the visible light range and over a plurality of frames.

In the specific case where alternately visible light and non-visible light (usually NIR FI light) are used, with two separate but correlated channels, which are correlated in spatial-and change-overarching fashion, it is the same scene that is recorded, but registered in different spectra. The time offset is minor in this case. The weaker non-visible information, is preferably not used to track a change during the recording of the images since this alone would be unnecessarily inaccurate, too weak, blurred and contain too few details.

The movement correlation to the white light is now transferred by extrapolated movement compensation data from the visible light to the non-visible light and is used for the required image optimization in the non-visible light. In endoscopic observations, the movement compensation was found to be very effective as movements generally have an overall uniformity on account of the relatively sluggish mass of the endoscopic system and, often, their additional attachment to holding elements. The image optimization is applied in a movement-compensated manner over a plurality of past frames on the basis of the change in the images in the visible light range described by white light vectors, to the images in the non-visible light range.

It should be noted it is commonly the case that the first illumination and the second illumination are provided by different light sources, however in some embodiments they may be provided by a single light source. In the latter case, use can be made of a switchable filter for example, which for an excitation frame blocks most white light, passing only the excitation wavelength of the fluorophore, and passes white light for the visible frame. However, it is also possible to dispense with such a filter for the light source if two image sensors are used for the image recording, one sensor of which is substantially sensitive to visible light while the other one is substantially sensitive to non-visible light. However, in principle, it is also possible to use only one image sensor if the filter is connected in front thereof such that either visible light or non-visible light is substantially guided to the image sensor.

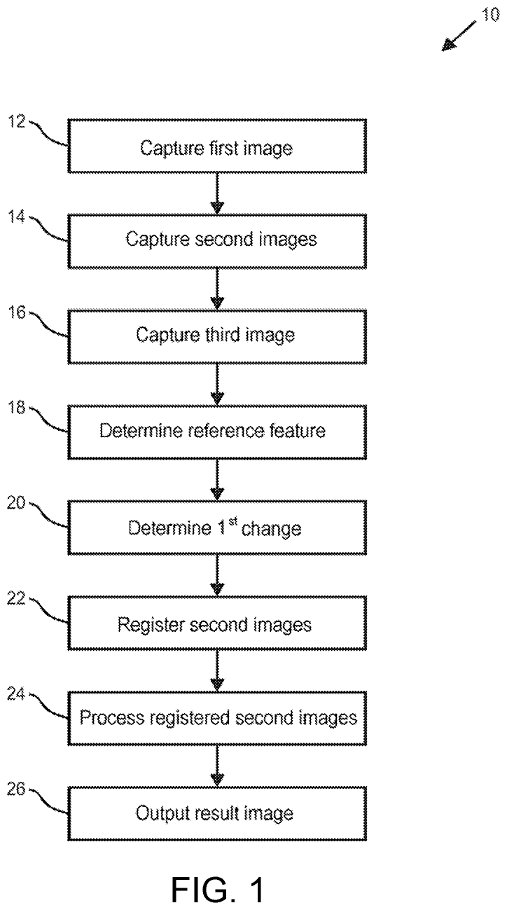

FIG. 1 shows a first embodiment of an imaging method 10 for imaging a scene 110 (see FIG. 10) during a change of a field of view 112 (see FIG. 10) of an image sensor 114 (see FIG. 10) relative to the scene 110. The steps of the method 10 are described below.

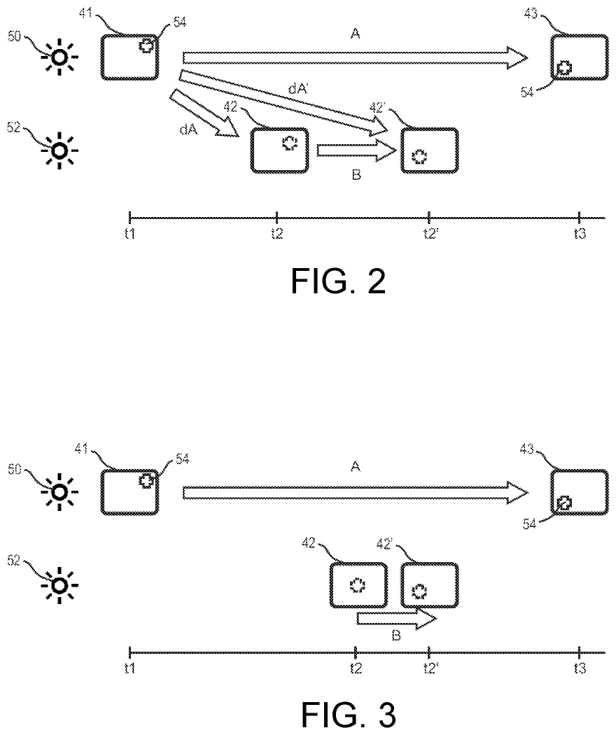

In a first step 12, a first image 41 (see FIG. 2) in the scene is captured with a first illumination 50 (see FIG. 2) of the scene 110 at a first time t1 (see FIG. 2), with the first image 41 predominantly containing first image information from the scene 110 in a visible light range.

In step 14, which follows step 12, a plurality of second images 42, 42′ (see FIG. 2) in the scene 110 are captured with a second illumination 52 (see FIG. 2) of the scene 110 at a respective second time t2, t2′ (see FIG. 2), with the second images 42, 42′ predominantly containing second image information from the scene in a non-visible light range.

In step 16, which follows step 14, a third image 43 in the scene 110 is captured with the first illumination 50 of the scene 110 at a third time t3 (see FIG. 2), with the third image 43 predominantly containing third image information from the scene 110 in the visible light range.

In step 18, at least one reference feature 54 (see FIG. 2) is determined in the scene 110, which reference feature is imaged in the first and in the third image 41, 43. FIG. 2 symbolically depicts that the reference feature 54 is also contained in the second images 42, 42′. However, as the feature can usually only be recognized unsharply in the images, it is not particularly suitable for a registration of the second images 42, 42′ on the basis of the image information contained in the second images 42, 42′ alone.

In step 20, a change A of the field of view 112 is determined on the basis of the at least one reference feature 54. In the case of the first chronological sequence of the recording of images in accordance with FIG. 2, the change A is represented simply as a translation. However, as already discussed, the change may include one or more elements from a group including translation, rotation, tilt, change of an optical focal length or change of a digital magnification.

In step 22, the second images 42, 42′ are, while considering at least one second change B that arises as a first proportion of the first change A, with the first proportion being the ratio of a first time difference between the second times t2, t2′ of two second images 42, 42′ to be registered and a second time difference between the third time t3 and the first time t1. This can be expressed as a formula as follows: B=A*(t2′−t2)/(t3−t1).

Another approach in this embodiment is as follows: For each second image 42, 42′ of the second images 42, 42′, the change A of the field of view 112 is interpolated to the second time t2, t2′ assigned to the second image 42, 42′ in order to obtain a partial change dA, dA′ of the field of view 112 for the second image 42, 42′. In particular, this partial change dA can be calculated as dA=A*(t2−t1)/(t3−t1) and dA′=A*(t2′−t1)/(t3−t1). Moreover, the second change B between the second images 42, 42′ can then be calculated as B=dA′−dA. Then, B=A*(t2′−t2)/(t3−t1) is also true.

In a step 24, the second images 42, 42′ are registered to bring the second images 42, 42′ into correspondence while considering the second change B, or alternatively the respective obtained partial changes dA, dA′, and thus to obtain registered second images.

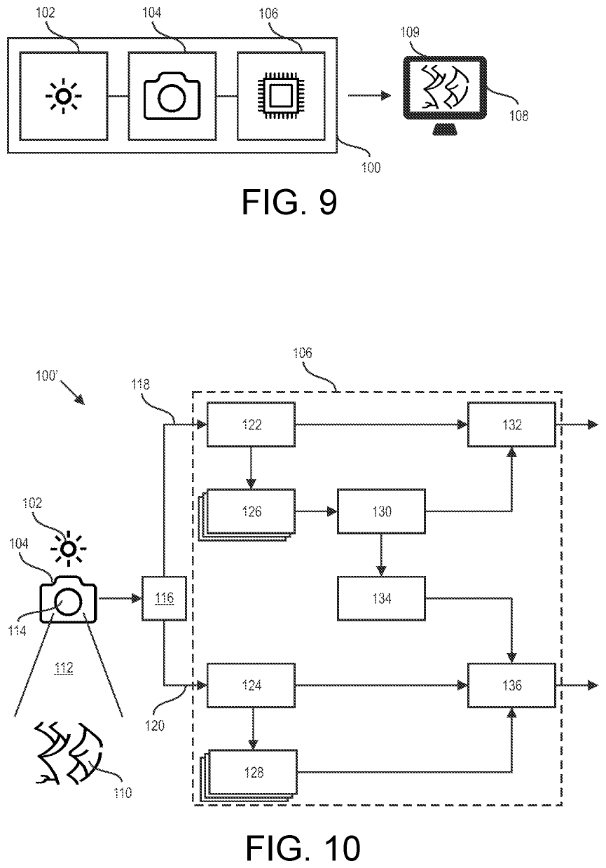

In a step 26, the registered second images are processed in order to obtain a resultant image 109 (see FIG. 9). Thus, for example, the brightness values of the registered second images can be added in order to increase the contrast. While a longer exposure time might have been used in the prior art, leading to a blurring of contours. By contrast, the processing of the registered second images leads to a better result image without blurring the contours.

In a step 28, the resultant image is output to a monitor 108 (see FIG. 9), where it is displayed. Optionally, the result can be output as such or together with the first and/or third image 41, 43 in the overlay.

FIG. 2 shows a first chronological sequence for recording images. In this case, a first image 41 is recorded at a first time t1, a first-second image 42 is recorded at a first-second time t2, a second-second image 42′ is recorded at a second-second time t2′ and a third image 43 is recorded at a third time t3. It should be noted that three, four, five or more second images 42 can also be captured between the first image 41 and the third image 43.

The movement of the reference feature 54 is used to symbolically depict how the field of view 112 of the image sensor 114 changes relative to the scene 110. A change A arises between the first image 41 and the third image 43, a change dA arises between the first image 41 and the first-second image 42, a change dA′ arises between the first image 41 and the second-second image 42′ and a second change B arises between the first-second image 42 and the second-second image 42′.

FIG. 3 shows a second chronological sequence for recording images. The same explanations as for FIG. 2 apply, with the symbolic arrows for the changes dA and dA′ having been omitted to improve clarity. It is possible to identify that the invention supports any temporal positioning of the second images 42, 42′.

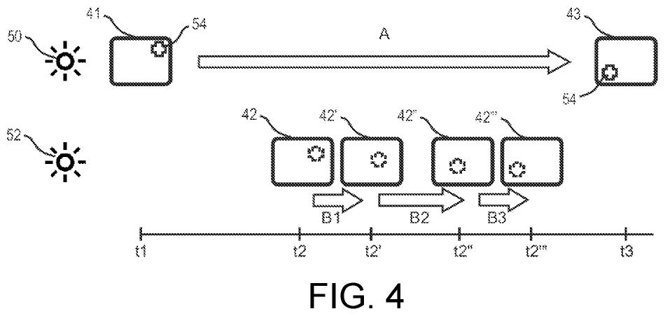

FIG. 4 shows a third chronological sequence for recording images. The same explanations as previously apply. In contrast to FIGS. 2 and 3, four second images 42, 42′, 42″, 42′″ are recorded in this case. A plurality of second changes can be determined in this case: B1=A*(t2′−t2)/(t3−t1), B2=A*(t2″−t2′)/(t3−t1) and B3=A*(t2′″−t2″)/(t3−t1). Hence, there is the option of registering all four second images 42, 42′, 42″, 42′″ to one another.

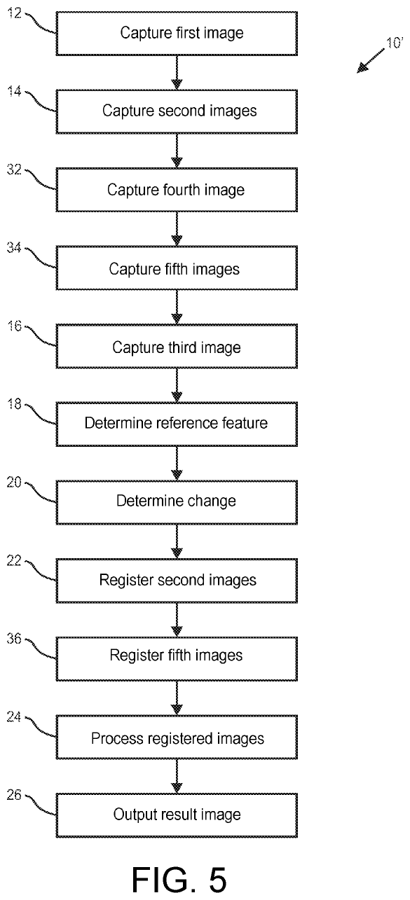

FIG. 5 shows a second embodiment of an imaging method 10′ for imaging a scene 110 during a change of a field of view 112 of an image sensor 114 relative to the scene 110. The steps of the method 10′ are explained below, with only the differences from the method 10 of FIG. 1 being discussed.

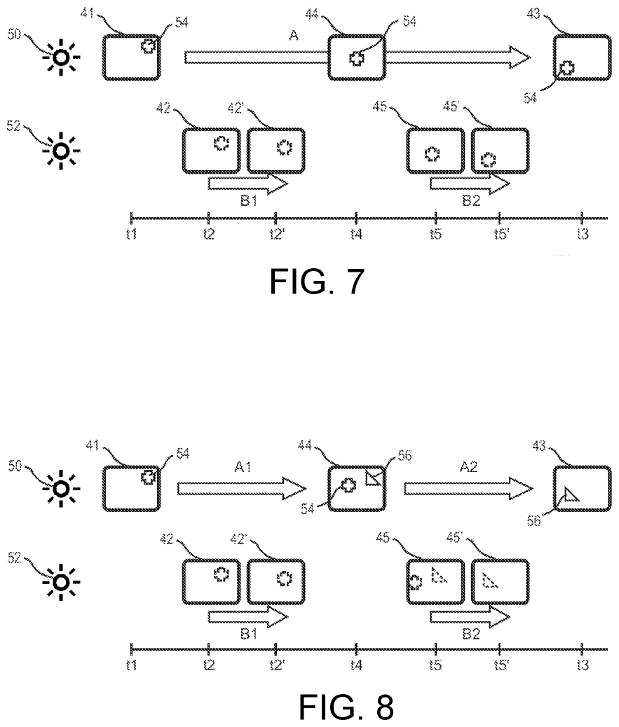

In a step 32, a fourth image 44 (see FIG. 7) in the scene 110 is captured with the first illumination 50 of the scene 110 at a fourth time t4, with the fourth image 44 predominantly containing fourth image information from the scene 110 in the visible light range.

In a step 34 a plurality of fifth images 45, 45′ in the scene 110 are captured with the second illumination 52 of the scene 110 at a respective fifth time t5, with the fifth images 45, 45′ predominantly containing fifth image information from the scene 110 in the non-visible light range.

In a step 36, the fifth images 45, 45′ are while considering at least one third change B2 (see FIG. 7), which arises as a second proportion of the first change A, with the second proportion being the ratio of a third time difference between the fifth times t5, t5′ of two fifth images 45, 45′ to be registered and the second time difference between the third time t3 and the first time t1. In one embodiment, this can be expressed as a formula as follows: B2=A*(t5′−t5)/(t3−t1).

In step 24, both the registered second images and the registered fifth images are processed in order to obtain a resultant image.

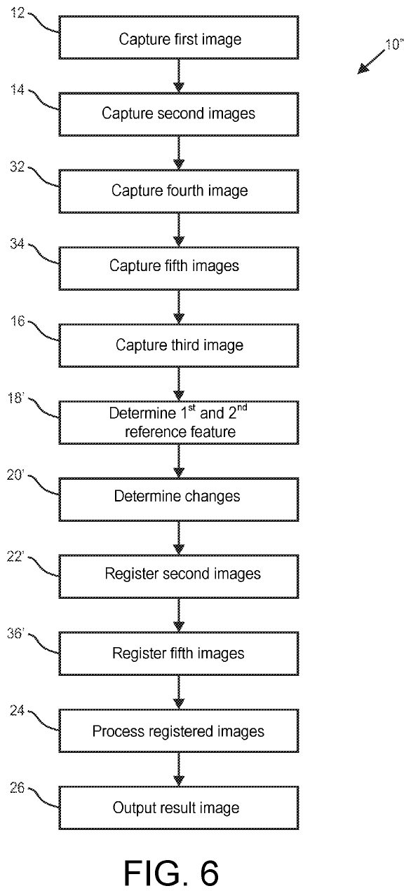

FIG. 6 shows a third embodiment of an imaging method 10″ for imaging a scene 110 during a change of a field of view 112 of an image sensor 114 relative to the scene 110. The steps of the method 10″ are explained below, with only the differences from the method 10′ of FIG. 5 being discussed.

Instead of step 18, a step 18′ is now carried out, in which at least one first reference feature 54 is determined in the scene 110, which reference feature is imaged in the first and in the fourth image 41, 44. Moreover, at least one second reference feature 56 is determined in the scene 110, which reference feature is imaged in the fourth and in the third image 44, 43. It is possible for the first reference feature 54 to be the same as the second reference feature 56. However, the first reference feature 54 may also differ from the second reference feature 56.

Instead of step 20, a step 20′ is now carried out, in which a first change A1 of the field of view 112 is determined on the basis of the at least one first reference feature 54. Moreover, a further change A2 of the field of view 112 is determined on the basis of the at least one second reference feature 56.

Instead of step 22, a step 22′ is now carried out, in which the second images 42, 42′ are registered while considering at least one second change B 1, which arises as a first proportion of the first change A1, with the first proportion being the ratio of a first time difference between the second times t2, t2′ of two second images 42, 42′ to be registered and a second time difference between the fourth time t4 and the first time t1. In one embodiment, this can be expressed as a formula as follows: B1=A*(t2′−t2)/(t4−t1).

Instead of step 36, a step 36′ is now carried out, in which the fifth images 45, 45′ while considering at least one third change B2, which arises as a second proportion of the further change A2, with the second proportion being the ratio of a third time difference between the fifth times t5, t5′ of two fifth images 45, 45′ to be registered and a fourth time difference between the third time t3 and the fourth time t4. In one embodiment, this can be expressed as a formula as follows: B2=A*(t5′−t5)/(t3−t4).

FIG. 7 shows a fourth chronological sequence for recording images. The same explanations as previously apply. In this case, a first image 41 is recorded at a first time t1, a first second image 42 is recorded at a first second time t2, a second-second image 42′ is recorded at a second-second time t2′, a fourth image 44 is recorded at a fourth time t4, a first-fifth image 45 is recorded at a first-fifth time t5, a second-fifth image 45′ is recorded at a second-fifth time t5′ and a third image 43 is recorded at a third time t3. Attention is drawn to the fact that three, four, five or more second images 42 may also be produced between the first image 41 and the fourth image 44 and/or that three, four, five or more fifth images 45 may be produced between the fourth image 44 and the third image 43.

FIG. 8 shows a fifth chronological sequence for recording images. The same explanations as for FIG. 7 apply. In contrast to FIG. 7, the fourth image 44 is used as an additional supporting image in this case, and so the registration of the second and fifth images 42, 42′, 45, 45′ is not implemented on the basis of the change A but implemented separately. Specifically, the registration of the second images 42, 42′ is implemented on the basis of the first change A1 and the registration of the second images 42, 42′ is implemented on the basis of the further change A2.

FIG. 9 shows a first embodiment of a system 100 for imaging a scene 110 during a change of a field of view 112 of an image sensor 114 relative to the scene 110. The system 100 comprises at least one illumination device 102, at least one imaging apparatus 104 and a processing device 106. The processing device 106 is designed to cause the system 100 to carry out one of the above-described methods 10, 10′, 10″.

FIG. 10 shows a second embodiment of a system 100′ for imaging a scene 110 during a change of a field of view 112 of an image sensor 114 relative to the scene 110. The system 100′ comprises at least one illumination device 102, at least one imaging apparatus 104 and a processing device 106. The imaging apparatus 104 feeds the recorded image stream to a switch 116, which transmits an image stream with images from the visible light range to a first processing path 118 and transmits an image stream with images from the non-visible light range to a second processing path 120. The image streams are, in each case correspondingly, divided into individual images or frames in a segmentation apparatus 122 or 124. The frames are, in each case correspondingly, stored in a memory apparatus 126 or 128 for further processing. The change A (or the changes A1 and A2) is (or are) calculated or estimated in a calculation device 130 from the frames in the visible light range. This information is used to register the frames in the visible light range in a compensation apparatus 132. By way of example, this can counteract blurring. The determined change A (or the changes A1 and A2) is (or are) used, as explained in conjunction with the preceding figures, to determine in an extrapolation and/or interpolation apparatus 134 how the determined change A (or the changes A1 and A2) has (or have) had an effect on the images in the non-visible light range. This information, specifically the second change B (or the changes B1 and B2) is (or are) used to register the frames in the non-visible light range in a compensation apparatus 136. By way of example, this can counteract blurring even if it is not possible to take a sufficient amount of information from the images in the non-visible light range in order to register these.

Although the invention and its advantages have been described in detail, it should be understood that various changes, substitutions and alterations can be made herein without departing from the scope of the invention as defined by the appended claims. The combinations of features described herein should not be interpreted to be limiting, and the features herein may be used in any working combination or sub-combination according to the invention. This description should therefore be interpreted as providing written support, under U.S. patent law and any relevant foreign patent laws, for any working combination or some sub-combination of the features herein.

Moreover, the scope of the present application is not intended to be limited to the particular embodiments of the process, machine, manufacture, composition of matter, means, methods and steps described in the specification. As one of ordinary skill in the art will readily appreciate from the disclosure of the invention, processes, machines, manufacture, compositions of matter, means, methods, or steps, presently existing or later to be developed that perform substantially the same function or achieve substantially the same result as the corresponding embodiments described herein may be utilized according to the invention. Accordingly, the appended claims are intended to include within their scope such processes, machines, manufacture, compositions of matter, means, methods, or steps.

1. An imaging method for imaging a scene during a change of a field of view of an image sensor relative to the scene, the method including the following steps:
a) capturing a first image in the scene with a first illumination of the scene at a first time (t1), the first image predominantly containing first image information from the scene in a visible light range,
b) following step a, capturing a plurality of second images in the scene with a second illumination of the scene at a respective second time (t2, t2′), the second images predominantly containing second image information from the scene in a non-visible light range,
c) following step b, capturing a third image in the scene with the first illumination of the scene at a third time (t3), with the third image predominantly containing third image information from the scene in the visible light range,
d) determining at least one reference feature in the scene, the reference feature imaged in the first and in the third image,
e) determining a first change (A) of the field of view on the basis of the at least one reference feature,
f) registering the second images while considering at least one second change (B), which arises as a first proportion of the first change (A), with the first proportion being the ratio of a first time difference between the second times (t2, t2′) of two second images to be registered and a second time difference between the third time (t3) and the first time (t1),
g) processing the registered second images in order to obtain a resultant image, and
h) outputting the resultant image.

2. The imaging method of claim 1, wherein the method includes the further steps of:
i) following step b and before step c, capturing a fourth image in the scene with the first illumination of the scene at a fourth time, the fourth image predominantly containing fourth image information from the scene in the visible light range,
j) following step i and before step c, capturing a plurality of fifth images in the scene with the second illumination of the scene at a respective fifth time, the fifth images predominantly containing fifth image information from the scene in the non-visible light range,
k) following step e and before step g, registering the fifth images while considering at least one third change, that arises as a second proportion of the first change, with the second proportion being the ratio of a third time difference between the fifth times of two fifth images to be registered and the second time difference between the third time and the first time, and
with the registered second images and the registered fifth images being processed in step g in order to obtain a resultant image.

3. The imaging method of claim 2, wherein step d is instead carried out as follows: determining at least one first reference feature in the scene, the first reference feature is imaged in the first and in the fourth image, and at least one second reference feature in the scene, the second reference feature is imaged in the fourth and in the third image;
wherein step e is instead carried out as follows: determining a first change (A1) of the field of view on the basis of the at least one first reference feature and a further change (A2) of the field of view on the basis of the at least one second reference feature;
wherein step f is instead carried out as follows: registering the second images while considering at least one second change (B1), that arises as a first proportion of the first change (A1), the first proportion being the ratio of a first time difference between the second times (t2, t2′) of two second images to be registered and a second time difference between the fourth time (t4) and the first time (t1); and
wherein step k is instead carried out as follows: registering the fifth images while considering at least one third change (B2), which arises as a second proportion of the further change (A2), with the second proportion being the ratio of a third time difference between the fifth times (t5, t5′) of two fifth images to be registered and a fourth time difference between the third time (t3) and the fourth time (t4).

4. The imaging method of claim 3, wherein a first intensity of the first image and of the third image is greater in each case than a second intensity of each of the second images.

5. The imaging method of claim 1, wherein a first intensity of the first image and of the third image is greater in each case than a second intensity of each of the second images.

6. The imaging method of claim 2, wherein a first intensity of the first image and of the third image is greater in each case than a second intensity of each of the second images.

7. The imaging method of claim 1, wherein the second images predominantly contain second image information from the scene in a near infrared range.

8. The imaging method of claim 2, wherein the second images predominantly contain second image information from the scene in a near infrared range.

9. The imaging method of claim 3, wherein the second images predominantly contain second image information from the scene in a near infrared range.

10. The imaging method of claim 4, wherein the second images predominantly contain second image information from the scene in a near infrared range.

11. The imaging method of claim 5, wherein the second images predominantly contain second image information from the scene in a near infrared range.

12. The imaging method of claim 6, wherein the second images predominantly contain second image information from the scene in a near infrared range.

13. The imaging method of claim 1, wherein the second images are brought into correspondence with a reference image.

14. The imaging method of claim 7, wherein the second images are brought into correspondence with a reference image.

15. The imaging method of claim 8, wherein the second images are brought into correspondence with a reference image.

16. The imaging method of claim 9, wherein the second images are brought into correspondence with a reference image.

17. The imaging method of claim 1, wherein the processing of the second registered images includes the application of computational photography.

18. The imaging method of claim 2, wherein the processing of the second registered images includes the application of computational photography.

19. The imaging method of claim 3, wherein the processing of the second registered images includes the application of computational photography.

20. A system for imaging a scene during a change of a field of view of an image sensor relative to the scene, the system comprising:
at least one illumination device,
at least one imaging apparatus,
a processing device which is designed to cause the system to carry out a method according to claim 1.
